(19) United States
(12) Patent Application Publication
(10) Pub. No.: US 2026/0013800 A1
(43) Pub. Date: Jan. 15, 2026

(54) Computer implemented Methods for Predicting Glucose Values, Data Processing, and App

(71) Applicants: Roche Diabetes Care, Inc., Indianapolis, IN (US); International Business Machines Corporation, Armonk, NY (US)

(72) Inventors: Paul J. Galley, Cumberland, IN (US); Yannick Klopfenstein, Neuchatel (CH); Eemeli Leppaaho, Espoo (CH); Patrick Lustenberger, Herrliberg (CH); Ajandek Peak, Munchen (DE); Christian Ringemann, Mannheim (DE)

(73) Assignees: Roche Diabetes Care, Inc., Indianapolis, IN (US); International Business Machines Corporation, Armonk, NY (US)

(21) Appl. No.: 19/336,870

(22) Filed: Sep. 23, 2025

Related U.S. Application Data
(63) Continuation of application No. PCT/US2023/017080, filed on Mar. 31, 2023.

(51) Int. Cl. A61B 5/00; A61B 5/145

(52) U.S. Cl. CPC A61B 5/7282; A61B 5/14532; A61B 5/742; A61B 5/7475

(57) ABSTRACT

A computer-implemented method and system for predicting and displaying glucose values, including receiving CGM data, determining, based on the data, a plurality of first predicted glucose values (33) for a first prediction time window (30), determining, based on the data, that a hypoglycemia event is predicted to occur during a second prediction time window (31) which has a contemporaneous beginning with the first prediction time window (30) but is shorter than the first window (30), and determining a plurality of second predicted glucose values (34) for the second prediction time window (31) and displaying the plurality of second predicted glucose values (34) for the second prediction time window (31) while not displaying predicted glucose values subsequent to the second prediction time window (31).

### CROSS-REFERENCE TO RELATED APPLICATIONS

This application claims priority to PCT application number PCT/US2023/017080, filed Mar. 31, 2023. The full disclosure of PCT application number PCT/US2023/017080 is incorporated by reference herein.

The present disclosure relates to computer-implemented methods for predicting glucose values. Further, the present disclosure refers to a data processing system for predicting glucose values, and an app.

### BACKGROUND

People may suffer from either Type I or Type II diabetes, in which the sugar level in the blood is not properly regulated by the body. Many of these people may use continuous glucose monitoring (CGM) to monitor their glucose level on an ongoing basis. In order to perform CGM, a glucose sensor may be placed under the skin of a person having diabetes for measuring the glucose level in the interstitial fluid. The glucose sensor may periodically measure the glucose level, such as once a minute, and transmit the results of the glucose measurement result to an insulin pump, blood glucose meter, smart phone or other electronic monitor. For predicting glucose values, typically static prediction time windows are employed.

Advances in medical technology have resulted in medical devices that allow patients to manage their medical conditions with relative ease. For example, several decades ago, a patient suffering from diabetes would have found it difficult to monitor and control the level of glucose in his bloodstream on his own. Today, however, a patient suffering from diabetes can monitor the level of glucose in his bloodstream using a portable blood glucose monitor and, if needed, administer a dosage of insulin to control the level of glucose in his bloodstream.

Some blood glucose monitors, such as a continuous glucose monitor (CGM), continuously generate raw data which corresponds to the level of glucose in a patient's bloodstream. Some CGMs transmit the generated data to a diabetes management device. Other devices, such as an insulin infusion pump, can also generate data and transmit the data to the diabetes management device. The sheer amount of data generated by the CGM and received by the diabetes management device can overwhelm a patient who is using the diabetes management device. Thus, there is a need to present the data in a more manageable manner.

As set out in document US 2022/0061712 A1, data describing glucose measurements are received from a continuous glucose monitoring system worn by a user and predicted glucose values during a future time period are generated for the user based on the data. A determination is made that at least one of the predicted glucose values satisfies a threshold value for an alert, which is associated with a prediction horizon that defines an amount of time prior to satisfaction of the threshold value for communicating the alert to the user.

Document US 2020/0098464 A1 discloses a method of monitoring a physiological condition of a patient that involves obtaining data indicative of a current state of the patient, identifying one or more historical patient states similar to the current state of the patient based on historical data associated with the one or more historical patient states maintained in a database, and obtaining a model for the physiological condition of the patient in the future from the current state. The model is determined based on the historical data associated with the one or more historical patient states.

In document U.S. Pat. No. 11,229,406 B2, a method of monitoring a physiological condition of a patient is described that involves obtaining current measurement data for the physiological condition of the patient provided by a sensing arrangement, obtaining a user input indicative of future events associated with the patient, and in response to the user input, determining a prediction of the physiological condition of the patient in the future based on the current measurement data and the future events using one or more prediction models associated with the patient.

Document US 2017/0177825 A1 discloses a method of determining a level of hypoglycemic unawareness displayed by a patient that includes maintaining a data structure including one or more glucose concentrations, receiving a glucose concentration, and determining a query based upon the received glucose concentration and the data structure.

### SUMMARY

It is an object of the present disclosure to provide improved technologies for predicting and displaying glucose values of a person having diabetes.

For solving the problem, computer-implemented methods for predicting glucose values as well as a data processing system, and an app (i.e., a computer program product) are provided according to the independent claims. Further embodiments are disclosed in the dependent claims and detailed below.

According to one aspect, a computer-implemented method for predicting and displaying glucose values is provided where the method is carried out in a system comprising at least one data processing device. The method comprises receiving continuous glucose monitoring data indicative of a glucose level in a bodily fluid from a continuous glucose monitoring system worn by a user; receiving the user's carbohydrate ingestion data and insulin administration data; determining, based on the continuous glucose monitoring data, the carbohydrate ingestion data and the insulin administration data, a plurality of first predicted glucose values for a first prediction time window; determining, based on the continuous glucose monitoring data and the carbohydrate ingestion data that a hypoglycemia event is predicted to occur during a second prediction time window which has a contemporaneous beginning with the first prediction time window but is shorter than the first prediction time window; and determining a plurality of second predicted glucose values for the second prediction time window and displaying the plurality of second predicted glucose values for the second prediction time window while not displaying predicted glucose values subsequent to the second prediction time window.

According to another aspect, a data processing system for predicting glucose values is provided. The system comprises at least one processor of a data processing device and is configured to receive continuous glucose monitoring data indicative of a glucose level in a bodily fluid from a continuous glucose monitoring system worn by a user; receive the user's carbohydrate ingestion data and insulin administration data; determine, based on the continuous glucose monitoring data, the carbohydrate ingestion data and the insulin administration data, a plurality of first predicted glucose values for a first prediction time window; determine, based on the continuous glucose monitoring data and the carbohydrate ingestion data that a hypoglycemia event is predicted to occur during a second prediction time window which has a contemporaneous beginning with the first prediction time window but is shorter than the first prediction time window; and determine a plurality of second predicted glucose values for the second prediction time window and display the plurality of second predicted glucose values for the second prediction time window while not displaying predicted glucose values subsequent to the second prediction time window.

In another aspect, a computer-implemented method of rearranging a predicted analyte trend graph on a display of a computer system for viewing by a patient is provided. The method comprises receiving into a memory of the computer system continuous glucose monitoring data indicative of a glucose level in a bodily fluid from a continuous glucose monitoring system worn by a user; receiving into the memory the user's carbohydrate ingestion data and insulin administration data; determining, by a processor of the computer system, a first predicted analyte trend graph covering a first prediction time window, wherein the first predicted analyte trend graph comprises a plot of analyte measurement values over the first prediction time window; displaying the first predicted analyte trend graph on the display; determining, by the processor of the computer system, automatically and without user interaction using a rule-based algorithm, that a second predicted analyte trend graph shall be displayed on the display to the patient, the second predicted analyte trend graph covering a second prediction time window which has a contemporaneous beginning with the first prediction time window but is shorter than the first prediction time window, the second predicted analyte trend graph comprising a plot of analyte measurement values over the second prediction time window; and displaying on the display the second predicted analyte trend graph while not displaying predicted glucose values subsequent to the second prediction time window.

Further, an app (a computer program product) configured to provide a display of predicted analyte measurement values on a data processing device for viewing by a patient is provided, the app comprising instructions which, when the computer program is executed by a data processing device, cause the data processing device to carry out the method for predicting glucose values.

The first and/or the second prediction time window may start from a current time (present time) and/or extend to future times (for which glucose values may be predicted). In other words, the second prediction time window has a contemporaneous beginning with the first prediction time window but is shorter than the first prediction time window. The duration of the first prediction time window may be predetermined. For example, the first prediction time window may have a length of time between 45 minutes and 360 minutes, preferably between 60 minutes and 180 minutes.

The determination that a hypoglycemic risk is predicted to occur may be determined based on a probability that a hypoglycemic event will occur during the second prediction time window exceeding a predetermined probability threshold. A hypoglycemic risk algorithm may be utilized for this probability determination, and the hypoglycemic risk algorithm would typically determine the risk that the user will have a hypoglycemic event within the second prediction time window, which as noted above has a contemporaneous beginning with the first prediction time window but is shorter than the first prediction time window. In one embodiment, the second prediction time window is set to be a length of time between 15 minutes and 45 minutes, preferably 30 minutes. In an embodiment, this algorithm can take into account the user's historical data (the user's past data). As an example, this algorithm can take into account the user's CGM data and their carbohydrate intake (i.e., carbohydrate ingestion) data. Optionally, the algorithm can take into account the user's insulin administration data and/or their activity data (e.g., their biometric data).

The probability threshold may be, e.g., at least 50%, preferably at least 75%, more preferably at least 90%. For example, if the probability of a hypoglycemic event within the first prediction time window exceeds 90%, the system and method described herein would call for the user to be shown a shorter duration time window of predicted glucose values as described herein.

The above-described determining of the plurality of second predicted glucose values for the second prediction time window and displaying the plurality of second predicted glucose values for the second prediction time window while not displaying predicted glucose values subsequent to the second prediction time window can be done in one embodiment by simply selecting the first predicted glucose values for the timeframe covered by the second prediction time window and denoting those values to be the plurality of second predicted glucose values. In other words, the predicted glucose values for the second prediction time window can be set by forming a subset of the first predicted glucose values, namely by simply truncating the previously determined first predicted glucose values to use only those predicted glucose values for the timeframe of the second prediction time window (which as noted above has a contemporaneous beginning with the first prediction time window but is shorter than the first prediction time window).

As a result of the described embodiments, it may for example be avoided that if the person with diabetes is close to a critical hypoglycemic condition, they might not take action in case the predicted glucose values incorrectly suggest a natural recovery from hypoglycemia. For example, in the described embodiments, by displaying to the user a shorter prediction window of future glucose predicted values (the above-described “second prediction time window”), the user's attention is thereby drawn to the fact that a hypoglycemic event is predicted and thus, can easily see that they need to take action. In other words, if the hypoglycemic risk algorithm determines that the user will likely have a hypoglycemic event within the second prediction time window, the system will only show the user a glucose prediction for the time encompassed by the second prediction time window, e.g., 30 minutes. In another illustrative embodiment, the system can be configured to only show the user a glucose prediction for a time as short as 15 minutes provided a high hypo risk situation persists to give the user time to react to rectify their hypoglycemic risk.

Static prediction time windows for displaying predicted glucose values to a user can be an issue for users in the sense that their attention is not appropriately drawn to the importance of their current metabolic state if they are at risk of having a hypoglycemic event in the near future. By the method and system, models without a static prediction time window may be established that respond more appropriately in view of a predicted hypoglycemic event. When the glucose prediction time window is shortened, the person having diabetes has their attention drawn to the current situation and may take action sooner and not wait for the glucose curve to naturally resolve. Thus, if the person is close to a critical metabolic condition, the methods and systems provided herein may prevent the person from failing to take action because the person is not shown a full duration prediction time window which might incorrectly suggest to the person a natural recovery from hypoglycemia.

With respect to the method, the determining of a hypoglycemia event step can be carried out by a processor of the data processing device automatically and without user interaction using a rule-based algorithm.

With respect to the method, the second prediction time window can comprise a set amount of time.

With respect to the method, the second prediction time window can be settable by a user by using an input of the at least one data processing device.

With respect to the method, the second prediction time window can be set for 30 minutes.

With respect to the method, the first prediction time window can comprise a set amount of time.

With respect to the method, the determining of a hypoglycemia event step can be carried out by also considering the user's insulin administration data.

With respect to the system, the determining of a hypoglycemia event step can be carried out by a processor of the data processing device automatically and without user interaction using a rule-based algorithm.

With respect to the system, the second prediction time window can comprise a set amount of time.

With respect to the system, the second prediction time window can be settable by a user by using an input of the at least one processor.

With respect to the system, the second prediction time window can be set for 30 minutes.

With respect to the system, the first prediction time window can comprise a set amount of time.

With respect to the system, the system can be configured to determine that a hypoglycemia event is predicted to occur by also considering the user's insulin administration data.

An app can be configured to provide a display of predicted analyte measurement values on a data processing device for viewing by a patient, wherein the app comprises instructions which, when the app is executed by the data processing device, cause the data processing device to carry out one of the methods described above.

In an embodiment, the determining of the second prediction time window may comprise excluding a (first) time interval subsequent to an expected time of the at least one of the hypoglycemic events occurring, preferably from the first prediction time window.

In an embodiment, the determining of the second prediction time window may comprise restricting the second prediction time window to a confidence (time) interval around the expected time of the at least one of the glucose level influencing events occurring. The confidence interval may be a p confidence interval with p being at least 70%, preferably at least 85%, more preferably at least 95%.

The determining of the second prediction time window may also comprise restricting the second prediction time window to the confidence interval and an intermediate time interval from the present time to the starting time of the confidence interval (in case the confidence interval does not include the present time).

The method may comprise providing an alarm for hypoglycemia. Providing the alarm may depend on the first and/or second predicted glucose values. Providing the alarm, in particular an alarm intensity, may further depend on a hypoglycemia awareness of the person having diabetes. The alarm intensity may, e.g., correspond to a sound and/or light level. The hypoglycemia awareness may, e.g., be determined from historical data and/or be provided via user input.

The alarm (output) may be provided via an output device and/or an alarm device, e.g., a speaker and/or a display.

The plurality of second predicted glucose values for the second prediction time window may be displayed via the output device. Displaying the plurality of second predicted glucose values for the second prediction time window may comprise excluding a display of predicted glucose values outside the second prediction time window.

The probability that a hypoglycemic event will occur during the second prediction time window may be determined using a statistical model (e.g., a hypoglycemic event risk prediction algorithm) based on the continuous glucose monitoring data and from the user's historical data, typically including the user's carbohydrate consumption data. Optionally, the user's insulin administration data and further optionally the user's activity (e.g., biometric) data can also be considered.

The statistical model may be trained using historical data as training data, e.g., using a machine learning algorithm. The historical data may comprise times (of day) and/or dates of the glucose level influencing events (for example, meal consumption time stamps). The historical data may further comprise continuous glucose monitoring data, in particular, glucose values for the respective times before and subsequent to the glucose level influencing events. The statistical model may also be determined using linear regression.

The first predicted glucose values and/or second predicted glucose values may be determined using one or a plurality of prediction algorithms, preferably from the continuous glucose monitoring data, and typically from carbohydrate consumption and insulin administration data for the user and further optionally from the user's activity data. In particular, the first and/or second predicted glucose values may be determined using a plurality of prediction algorithms by determining a (weighted or unweighted) average of prediction values of each of the plurality of prediction algorithms. As should be clear from the above, a hypoglycemia event may be determined using algorithms by determining the (weighted or unweighted) average of prediction values of each of the plurality of prediction algorithms. The one or the plurality of prediction algorithms may be trained using the historical data as training data.

The first/second predicted glucose values may be determined using a physiological model for glucose level prediction. Prediction of the first/second predicted glucose values may or may not be dependent on the length of the first and/or second prediction time window. The first predicted glucose values and the second predicted glucose values may or may not coincide for overlapping times in the first and the second prediction time window.

The continuous glucose monitoring data may comprise glucose values. A glucose level and/or glucose values may be determined by continuous glucose monitoring via a fully or partially implanted sensor and/or worn sensor. In general, in the context of continuous glucose monitoring, a glucose value or glucose level in a bodily fluid may be determined. The glucose level or value may be, e.g., subcutaneously measured in an interstitial fluid. Continuous glucose monitoring may be implemented as a nearly real-time or quasi-continuous monitoring procedure frequently or automatically providing/updating analyte values without user interaction.

The embodiments described above in connection with the method for predicting glucose values may be provided correspondingly for the system for predicting glucose values. Within the context of the present disclosure, the indication of an interval using the terms “between . . . and . . . ” includes the limit points of the interval.

### BRIEF DESCRIPTION OF THE SEVERAL VIEWS OF THE DRAWINGS

In the following, embodiments, by way of example, are described with reference to figures.

FIG. 1 shows a graphical representation of a system for predicting glucose values.

FIG. 2 shows a graphical representation of a method for predicting glucose values.

FIG. 3 shows a graphical representation of a blood glucose trend graph with a first prediction time window illustrating a display presented to a user in a situation where the user is believed to have a low risk of hypoglycemia.

FIG. 4 shows a graphical representation of a blood glucose trend graph with a second prediction time window illustrating a display presented to a user in a situation where the user is believed to have a high risk of hypoglycemia.

### DETAILED DESCRIPTION

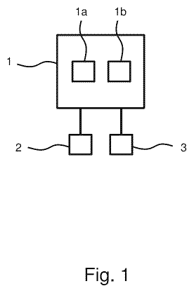

FIG. 1 shows a graphical representation of a system for predicting glucose values. The system comprises a data processing device 1 provided with one or more processors 1a and a memory 1b for storing machine readable instructions. The processing device 1 is connected to an input device 2 configured to receive (user) input data and an output device 3 configured for outputting data. The input device 2 and the output device 3 may or may not be implemented integrally with the data processing device 1.

The one or more processors 1a may be a controller, an integrated circuit, a microchip, a computer, or any other computing device capable of executing machine readable instructions. The memory 1b may be RAM, ROM, a flash memory, a hard drive, or any device capable of storing machine readable instructions.

The one or more processors 1a may be integral with a single component of the system. The one or more processors 1a may also be separately located within discrete components such as, for example, a glucose meter, a medication delivery device, a mobile phone, a portable digital assistant (PDA), a mobile computing device such as a laptop, a tablet, or a smart phone, a desktop computer, or a server, e.g., via a cloud or web-based technologies and communicatively coupled with one.

The output device 3 may be configured to provide graphical, textual and/or auditory information. The output device 3 may include an electronic display such as, for example, a liquid crystal display, thin film transistor display, light emitting diode display, a touch screen, or any other device capable of transforming signals from a processor into an optical output, or a mechanical output, such as, for example, a speaker or a printer for displaying information.

The input device 2 may comprise or be coupled to a body sensor for providing biological data indicative of properties of an analyte and/or a continuous glucose monitoring system coupled to a person having diabetes. The input device 2 may be configured to receive raw data from the body sensor and process the raw data into glucose monitoring data and, preferably, transmit the glucose monitoring data to the data processing device 1.

The body sensor may be a glucose sensor configured to detect glucose levels (e.g., glucose concentrations) when coupled to (in particular, worn by) a person having diabetes. For example, the body sensor can be a disposable glucose sensor that is, e.g., worn under the skin.

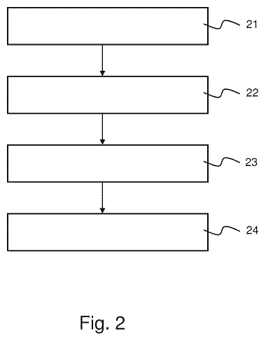

FIG. 2 shows a graphical representation of a method for predicting glucose values being carried out in the system with the (at least one) data processing device 1.

In a first step 21, continuous glucose monitoring data indicative of a glucose level in a bodily fluid are received in the data processing device 1 from the continuous glucose monitoring system coupled to the person having diabetes. The user's carbohydrate ingestion data and insulin administration data are also received in the data processing device 1. The user's carbohydrate ingestion data and insulin administration data can be logged manually by the user or received in the data processing device 1 in any other suitable way.

In a second step 22, a plurality of first predicted glucose values for a first prediction time window is determined based on the continuous glucose monitoring data, the carbohydrate ingestion data and the insulin administration data. The continuous glucose monitoring data, the carbohydrate ingestion data, the insulin administration data, and predicted glucose values may be stored in the memory 1b.

In a third step 23, this step includes determining, based on the user's continuous glucose monitoring data, carbohydrate ingestion data and optionally the user's insulin administration data (and optionally the user's activity data) that a hypoglycemia event is predicted to occur during a second prediction time window (31) which has a contemporaneous beginning with the first prediction time window (30) but is shorter than the first prediction time window (30). If a hypoglycemia event is predicted to occur during the second prediction time window (31), the user is alerted to the hypoglycemia event prediction by displaying to the user the second prediction time window glucose predictions rather than the glucose predictions for the longer first prediction time window. The time duration of the second prediction time window (31) may be factory set and/or modified by the user using the data processing device 1, e.g., via the input device 2.

A fourth step 24 calls for determining a plurality of second predicted glucose values (34) for the second prediction time window (31) and displaying the plurality of second predicted glucose values (34) for the second prediction time window (31) while not displaying predicted glucose values subsequent to the second prediction time window (31), and in one embodiment, while not displaying predicted glucose values subsequent to an expected time of the hypoglycemia event occurrence. The second predicted glucose values may be displayed to the person having diabetes, e.g., via the output device 3.

Disclosed herein is an algorithm which is run to determine whether the display presented to the user should show a full prediction horizon (e.g., a 2 hour prediction) or whether a more short-term prediction (e.g., a 30 minute prediction) should be shown given the user's current metabolic situation. The drawback of a prediction algorithm which presents a fixed timeframe for glucose predictions is that it will predict and display to the user the glucose concentration over that timeframe (e.g., the next two hours) irrespective of the current metabolic situation of the patient.

A shorter timeframe for future glucose prediction values is presented to a user if the system determines the user has a higher current glycemic risk to focus the user on their current risk and not introduce confusion with a longer prediction timeframe. While not wishing to be bound by theory, one benefit realized herein is that it is believed to be counterproductive or at worst misleading to focus the attention of the user to a longer time horizon (e.g., 2 hours) if their current metabolic condition calls for an immediate action (e.g., ingesting carbohydrates) and/or where the user's attention needs to be focused on their current condition.

The hypoglycemic event risk algorithm described herein can comprise a simple rule-based algorithm or a more sophisticated machine learning based algorithm that estimates the risk or probability of a higher risk user condition, e.g. a hypoglycemic event in the next e.g. 30 minutes, and then instead of showing a two hour prediction to the user, only a prediction over the next e.g. 30 minutes is shown. In an embodiment, an XGBoost model is utilized for this purpose. The term XGBoost stands for Extreme Gradient Boosting, and is a scalable, distributed gradient-boosted decision tree (GBDT) machine learning library. It provides parallel tree boosting and is a leading machine learning library for regression, classification, and ranking problems.

As an illustrative example, determining to present a shorter time frame for glucose predictions to the user can be based on the system receiving a predicted glucose value that is below a lower threshold (e.g., <70 mg/dl) or if the above-referenced hypoglycemic risk algorithm has identified a risk that such a glucose value or values will occur.

In another embodiment, a determination to present a shorter time frame for glucose predictions to a user can be based on the system receiving a predicted glucose value that is above a threshold (e.g., >200 mg/dl) or is merely elevated for a very long time (e.g., is higher than a target range for 2-3 hours).

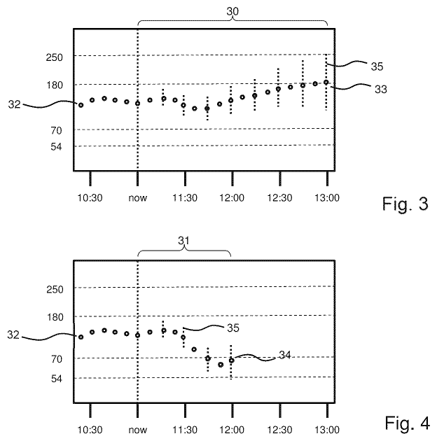

FIG. 3 illustrates an aspect of the subject matter in accordance with one embodiment. This figure illustrates an example of a potential screen display presented to a user in a situation where the user is believed to have a low risk of hypoglycemia. In this situation, as the user is believed to have a low risk of hypoglycemia, a first prediction time window including a full glucose prediction timeframe (e.g., 2 hours) is presented to the user.

FIG. 4 illustrates an aspect of the subject matter in accordance with one embodiment. This figure illustrates an example of a potential screen display presented to a user in a situation where the user is believed to have a high risk of hypoglycemia. In this situation, as the user is believed to have a high risk of hypoglycemia, a second prediction time window including a shorter glucose prediction timeframe (e.g., 30 minutes) is presented to the user rather than the full glucose prediction timeframe as shown in FIG. 3.

FIG. 3 shows a graphical representation of a blood glucose trend graph with a first prediction time window 30, and FIG. 4 shows a graphical representation of a blood glucose trend graph for a second prediction time window 31. The trend graphs comprise measured glucose values 32 (as part of the continuous glucose monitoring data), first predicted glucose values 33 for the first prediction time window 30, and second predicted glucose values 34 for the second prediction time window 31. As should be apparent from the above, the current metabolic situations reflected in FIGS. 3-4 differ from one another since FIG. 4 represents a higher risk metabolic situation (a risk of hypoglycemia) for the user. Confidence intervals for the predicted glucose values 33, 34 are represented by bars 35. The confidence intervals as shown by the bars 35 are larger for glucose predictions further out chronologically from the current time (the time labeled “now” in FIGS. 3-4).

The features disclosed in this specification, the figures and/or the claims may be material for the realization of various embodiments, taken in isolation or in various combinations thereof.

1. A computer-implemented method for predicting and displaying glucose values, the method being carried out in a system with at least one data processing device (1), the method comprising:
receiving continuous glucose monitoring data indicative of a glucose level in a bodily fluid from a continuous glucose monitoring system worn by a user;
receiving the user's carbohydrate ingestion data and insulin administration data;
determining, based on the continuous glucose monitoring data, the carbohydrate ingestion data and the insulin administration data, a plurality of first predicted glucose values (33) for a first prediction time window (30);
determining, based on the continuous glucose monitoring data and the carbohydrate ingestion data that a hypoglycemia event is predicted to occur during a second prediction time window (31) which has a contemporaneous beginning with the first prediction time window (30) but is shorter than the first prediction time window (30); and
determining a plurality of second predicted glucose values (34) for the second prediction time window (31) and displaying the plurality of second predicted glucose values (34) for the second prediction time window (31) while not displaying predicted glucose values subsequent to the second prediction time window (31).

2. The method of claim 1, wherein the determining of a hypoglycemia event step is carried out by a processor of the data processing device (1) automatically and without user interaction using a rule-based algorithm.

3. The method of claim 1, wherein the second prediction time window (31) comprises a set amount of time.

4. The method of claim 1, wherein the second prediction time window (31) is settable by a user by using an input of the at least one data processing device (1).

5. The method of claim 1, wherein the second prediction time window (31) is set for 30 minutes.

6. The method of claim 1, wherein the first prediction time window (30) comprises a set amount of time.

7. The method of claim 1, wherein the determining of a hypoglycemia event step is carried out by also considering the user's insulin administration data.

8. An app configured to provide a display of predicted analyte measurement values on a data processing device (1) for viewing by a patient, the app comprising instructions which, when the app is executed by a data processing device (1), cause a data processing device (1) to carry out the method of claim 1.

9. A data processing system for predicting glucose values, the system comprising at least one processor of a data processing device (1) and being configured to:
receive continuous glucose monitoring data indicative of a glucose level in a bodily fluid from a continuous glucose monitoring system worn by a user;
receive the user's carbohydrate ingestion data and insulin administration data;
determine, based on the continuous glucose monitoring data, the carbohydrate ingestion data and the insulin administration data, a plurality of first predicted glucose values (33) for a first prediction time window (30);
determine, based on the continuous glucose monitoring data and the carbohydrate ingestion data that a hypoglycemia event is predicted to occur during a second prediction time window (31) which has a contemporaneous beginning with the first prediction time window (30) but is shorter than the first prediction time window (30); and
determine a plurality of second predicted glucose values (34) for the second prediction time window (31) and display the plurality of second predicted glucose values (34) for the second prediction time window (31) while not displaying predicted glucose values subsequent to the second prediction time window (31).

10. The system of claim 9, wherein the determining of a hypoglycemia event is carried out by the processor automatically and without user interaction using a rule-based algorithm.

11. The system of claim 9, wherein the second prediction time window (31) comprises a set amount of time.

12. The system of claim 9, wherein the second prediction time window (31) is settable by a user by using an input of the at least one processor.

13. The system of claim 9, wherein the second prediction time window (31) is set for 30 minutes.

14. The system of claim 9, wherein the first prediction time window (30) comprises a set amount of time.

15. The system of claim 9, wherein the system is configured to determine that a hypoglycemia event is predicted to occur by also considering the user's insulin administration data.

16. A computer-implemented method of rearranging a predicted analyte trend graph on a display of a computer system for viewing by a patient, the method comprising:
receiving into a memory of the computer system continuous glucose monitoring data indicative of a glucose level in a bodily fluid from a continuous glucose monitoring system worn by a user;
receiving into the memory the user's carbohydrate ingestion data and insulin administration data;
determining, by a processor of the computer system, a first predicted analyte trend graph covering a first prediction time window, wherein the first predicted analyte trend graph comprises a plot of analyte measurement values (33) over the first prediction time window (30);
displaying the first predicted analyte trend graph on the display;
determining, by the processor of the computer system, automatically and without user interaction using a rule-based algorithm, that a second predicted analyte trend graph shall be displayed on the display to the patient, the second predicted analyte trend graph covering a second prediction time window (31) which has a contemporaneous beginning with the first prediction time window (30) but is shorter than the first prediction time window (30), the second predicted analyte trend graph comprising a plot of analyte measurement values (34) over the second prediction time window (31); and
displaying on the display the second predicted analyte trend graph while not displaying predicted glucose values subsequent to the second prediction time window (31).

17. An app configured to provide a display of predicted analyte measurement values on a data processing device (1) for viewing by a patient, the app comprising instructions which, when the app is executed by a data processing device (1), cause a data processing device (1) to carry out the method of claim 16.
